(19) United States
(12) Patent Application Publication
(10) Pub. No.: US 2014/0276475 A1
(43) Pub. Date: Sep. 18, 2014

(54) Bandage

(71) Applicant: John Richard Taylor, Arp, TX (US)

(72) Inventor: John Richard Taylor, Arp, TX (US)

(21) Appl. No.: 13/815,675

(22) Filed: Mar. 14, 2013

(51) Int. Cl. A61F 13/00; A61F 13/02

(52) U.S. Cl. CPC A61F 13/00063; A61F 13/0269
USPC 604/290; 604/304; 604/307

(57) ABSTRACT

An improved band-aid or bandage comprises a dry or dried medicament or medicinal substance in a gauze pad, medical dressing, or pouch. The gauze pad, medical dressing, or pouch is attached to a person's body with an attachment means that has an aperture therethrough, the aperture located adjacent the gauze pad, medical dressing, or pouch. A user can attach the band-aid or bandage to a body and then pass water or another liquid through the aperture to wet the dry or dried medicament or medicinal substance. Once wetted, the medicament or medicinal substance can then suffuse out of the gauze pad, medical dressing, or pouch and onto the part of the body to which it is adjacent, thereby obtaining a desired effect on the body. A method of delivering a medicament or medicinal substance comprises the steps of disposing a dry or dried medicament or medicinal substance in a gauze pad, medical dressing, or pouch; affixing the gauze pad, medical dressing, or pouch to an attachment means for attaching it to a body; attaching the gauze pad, medical dressing, or pouch to a body; and wetting the pouch with an amount of liquid sufficient to wet the dry or dried medicament or medicinal substance.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

(1) Field of Invention

This application relates to the field of medicament application devices. Medicament application devices can utilize a simple gauze pad of any useful size, shape, and thickness. The gauze pad is affixed to one of the two sides of a strip of tape. The tape usually is larger or longer than the gauze pad. One side of the tape has an adhesive disposed on some or all the surface of that side, and the gauze pad is affixed to the side with the adhesive. The other side of the tape usually does not have any adhesive. There may be plural relatively small holes through the tape to provide air flow from one side of the tape to the other. There may be one or more strips of cover tape that cover the adhesive-side of the tape and possibly the gauze pad, to prevent the adhesive on the tape from sticking to packaging or something else before tape is adhered in the manner desired.

(2) Description of Related Art

(a) Terminology

In the U.S., the device described above is commonly called a “bandage” or a “band-aid”. Technically, a “bandage” is a strip of cloth or other material used to bind up a wound, sore, or sprain. However, in the U.S., the common definition of “bandage” is now broader and includes the device described in the previous paragraph. It is still more common to call such a device a “band-aid” when the device is relatively small and is used for relatively minor wounds. Band-Aid was, at one time, a registered trademark in the U.S. and other countries. But, “band-aid” has since become a genericized trademark that is commonly used to refer to any brand of band-aid. The difference in terms seems to be that a “band-aid” refers to a relatively small device used mostly by non-medically-trained personnel for minor wounds, while a “bandage” refers to any such device from the smallest band-aid to relatively large bandages using surgical dressings instead of gauze pads and bindings that wrap completely around a part of the body to secure the surgical dressing to its proper site. The terms “band-aid” and “bandage” are used interchangeably in this application since size is not a significant consideration in this invention.

A medicinal substance or medicament is any curative or remedial substance used to treat any biological affliction. It would include not only a recognized disease or illness, but also a wound or minor affliction that does not require the attention of a medically-trained person. One example of a minor affliction is a common pimple on a person's skin. A medicinal substance or medicament may include a prophylactic substance that prevents infection or any other affliction from taking hold. The terms “medicament” and “medicinal substance” are used interchangeably in this application, since the invention contemplates the use of either.

(B) Prior Art

Medical dressings, including the relatively small gauze pad and the larger surgical dressings that are used in band-aids and bandages, have several purposes when applied to a wound to a body. They are used to stem bleeding, to absorb fluids exuded from the wound, to ease pain caused by the wound, to protect the wound from infection, to protect the wound from further mechanical damage, to promote healing, and sometimes to allow airflow across the wound which promotes healing. A band-aid or a bandage may utilize a gauze pad, a surgical dressing, or any other suitable medical dressing that tends to accomplish any or all of these functions.

The bandage or adhesive strip that holds the gauze pad in place over the wound will commonly be air permeable. For example, the bandage could be a simple cotton strip which is naturally air-permeable, or the bandage could be an adhesive plastic strip with small holes provided above the location of the gauze pad so as to allow air to enter the gauze pad and reach the wound. Wounds need to be protected from much of the environment during healing, but it is usually desirable that air reach the wound and move over it.

The gauze pad and the adhesive tape that holds the gauze pad to the body can both be made in any useful size, shape, or thickness. When the gauze pad is rectangular in shape, the adhesive tape can be arranged to extend beyond one, two, or more sides of the gauze pad. The adhesive tape may extend beyond the gauze pad in all directions regardless of the shape of the gauze pad.

A band-aid or a bandage can utilize any appropriate binding to bind the medical dressing to the body at the proper location and to keep it securely bound for an appropriate amount of time. A band-aid commonly comprises an elongated, relatively flat, strip of tape that has an adhesive spread along some or all of one flat side. Such a strip of tape may be made from cloth, plastic, or rubber. But a band-aid or a bandage can use any ligature as long as the medical dressing is securely held in the proper location for an appropriate amount of time. A ligature that binds a medical dressing can be made from a solid material, a gel, a liquid that hardens, or any suitable substance. The ligature is usually removable, but it may actually be designed to dissolve or come away over an appropriate time.

It is known to provide certain medicinal substances in the medical dressing of a band-aid or bandage that promote healing of the wound or inhibit infection. For example, anti-bacterial substances are known to be provided in the medical dressing. Mild anesthetics have also been provided in the medical dressing. However, these substances have been heretofore provided in a ready state. They perform their intended function as soon as the band-aid or bandage is applied to the wound without any further action on the part of a person.

A person's skin can suffer from various afflictions which can be treated by the application of various substances. For example, common pimples can be treated with inorganic salt compounds such as sodium chloride, sodium bicarbonate, and magnesium sulfate, if it is continuously applied to the pimple site for an extended time. Other afflictions can be treated with herbal remedies that should be continuously applied for an extended time. However, some remedies need to be in a liquid or semi-liquid form during the application period, but cannot be effectively or efficiently stored in a liquid or semi-liquid form in a medical dressing. It would be desirable if a substance could be placed in a medical dressing in a solid form, and then easily activated, reconstituted, or dissolved to a liquid or semi-liquid form at the time the medical dressing is placed on the afflicted site.

A band-aid or bandage can hold a medicinal substance in dry or dried form in the medical dressing. The medicinal substance in dry or dried form tends to be storable for an extended amount of time. It would be advantageous if, when a person desires to apply the medicinal substance to a part of a person's body, the dry or dried medicinal substance in the medical dressing can be activated or reconstituted, either just before the medical dressing is applied to the body or at some time afterwards. It is also possible to keep the dry or dried substance activated for an extended time by periodically applying water or another liquid to the medicinal substance when it dries. Further, the medical dressing may hold one substance in dry or dried form, and the infusion of another substance in wet form may cause the two substances to combine to produce a medicinal combination or substance.

A poultice is a soft moist mass of meal, herbs, or the like spread on a cloth and applied to a body as a medicament. It would be desirable if the medicament in a poultice could be stored in dry or dried form, then wetted at the time it is applied to the body and perhaps periodically during the application period.

U.S. Published Patent Application Number 2012/0283614 teaches a band-aid having a gel-pouch beneath the gauze. When an adhesive pull-tab is removed, an antiseptic gel or the like oozes out onto the gauze.

U.S. Pat. Nos. 6,555,729 and 6,770,794 teach a bandage having living larvae in a sealed pouch attached to an adhesive strip. A connector 22 provides a ventilation means for the larvae.

U.S. Pat. No. 4,117,841 teaches a medicated bandage adhesive strip with a pocket that can rupture and dispense a medicament.

U.S. Pat. No. 4,808,172 teaches an adhesive bandage having a capsule filled with a medicinal agent. The capsule can rupture and dispense the medicinal agent.

WO0202042, also published as U.S. Pat. No. 7,240,790, teaches a multiple-pouch bandage impregnated with a product for application on the skin and having a means for protecting the treated area.

WO2012088310 teaches a dual purpose adhesive bandage with an absorbent pad configured as a pouch having an open end for receiving a thermal pack. The bandage serves the purpose of a bandage and a thermal application pack.

### BRIEF SUMMARY OF THE INVENTION

A first improved bandage comprises an attachment means for attaching a medical dressing to a body, a medical dressing affixed to the attachment means, the medical dressing having a dry or dried medicament contained or impregnated in the medical dressing, and an aperture through the attachment means adjacent the medical dressing such that, after the medical dressing is attached to the body, a liquid can be passed through the aperture to the medical dressing to wet the medical dressing and the medicament therein. Alternatively, the dressing could be wetted before the bandage is attached to the body.

A second improved bandage comprises an attachment means for attaching a liquid-permeable pouch to a body, a pouch affixed to the attachment means, the pouch containing a dry or dried medicament, and an aperture through the attachment means adjacent the pouch such that, after the pouch is attached to the body, a liquid can be passed through the aperture to the pouch to wet the pouch and the medicament therein. Alternatively, the pouch could be wetted before it is attached to the body.

A third improved bandage comprises a first relatively larger piece of bandage material, a dry or dried medicament that is placed into a central portion of the first piece, a second relatively smaller piece of bandage material that is sealed onto the first piece such that all of the medicament is sandwiched between the first and second pieces of bandage material, and wherein an adhesive is disposed on at least one part of the first or second piece of bandage material such that the bandage can be attached to a body with the medicament adjacent the part of the body to be treated. Alternatively, the medicament could be placed into the second piece and then the first piece could be sealed onto the second piece. As another alternative, the two pieces could be approximately the same size and the seal formed around the perimeter of the medicament; the perimeter where the adhesive is applied would be two membranes and the perimeter would be somewhat stiffer. The first and/or second pieces of bandage material can then be wetted with a liquid sufficiently to activate or reconstitute the dry or dried medicament sandwiched between the first and second pieces of bandage material. The wetted and activated medicament would then suffuse through at least one of the first and/or second pieces of bandage material to come into contact with the part of the body to be treated. Activation or reconstitution can be caused either before or after the bandage is attached to the body.

Another possible use of any of the three improved bandages is that wetting or moistening of the bandage may allow the bandage to separate from a wound on the body more easily and/or with less trauma to the wound.

A first improved method of treating a part of a body comprises the steps of placing or impregnating a dry or dried medicament in a medical dressing, placing the medical dressing on the part of the body to be treated, attaching the medical dressing to the body with an attachment means having an aperture through the attachment means adjacent the medical dressing, and passing a liquid through the aperture in the attachment means to the medical dressing in a quantity sufficient to wet the medical dressing and the medicament therein. The medical dressing and medicament may be re-wetted periodically.

A second improved method of treating a part of a body comprises the steps of placing a dry or dried medicament into a pouch, placing the pouch on the part of the body to be treated, attaching the pouch to the body with an attachment means having an aperture through the attachment means adjacent the pouch, and passing a liquid through the aperture in the attachment means to the pouch in a quantity sufficient to wet the medical dressing and the medicament therein. The medical dressing and medicament may be re-wetted periodically.

A third improved method of making a bandage comprises the steps of placing a first relatively larger piece of bandage material onto a jig having a concave area in a relatively flat surface, placing a dry or dried medicament onto the first piece of bandage material in the area in which it lies in the concave area of the jig, placing a second relatively smaller piece of bandage material onto the first piece over the medicament and sealing the second piece to the first piece around the perimeter of the medicament, applying an adhesive to at least one part of at least one of the first and second pieces of bandage material such that the bandage can be attached to a body to be treated, and then wetting at least one of the first and second pieces with a liquid in a quantity sufficient to wet the dry or dried medicament contained in the bandage. The medical dressing and medicament may be re-wetted periodically.

The medical dressing and pouch may comprise a distribution means for causing the dry or dried medicament in the medical dressing or pouch to be distributed relatively uniformly throughout the medical dressing or pouch regardless of the orientation of the medical dressing or pouch with respect to gravity. This distribution means may comprise a baffle disposed within the medical dressing or pouch. Alternatively, the distribution means may comprise plural compartments formed within the medical dressing or pouch. Alternatively, the distribution means may comprise plural small pouches disposed within the pouch.

The liquid used for wetting and re-wetting may be one or more of water, a medicinal liquid, a catalyst liquid, or another suitable liquid.

### BRIEF DESCRIPTION OF THE SEVERAL VIEWS OF THE DRAWINGS

FIG. 1 is an isometric view of a first embodiment of this invention.

FIG. 2 is a top view of the first embodiment of this invention.

FIG. 3 is a bottom view of the first embodiment of this invention.

FIG. 4 is another top view of the first embodiment of this invention showing a pull-tab cover.

FIG. 5 is another top view of the first embodiment of this invention showing a perforated part of the adhesive strip.

FIG. 6 is a side view of a second embodiment of this invention.

FIG. 7 is a top view of the second embodiment of this invention.

FIG. 8 is a bottom view of the second embodiment of this invention.

FIG. 9 is another top view of the second embodiment of this invention showing a pull-tab cover.

FIG. 10 is a detail view showing a baffle within the second embodiment of this invention.

FIG. 11 is a detail view showing an alternative embodiment of the second embodiment of this invention.

FIG. 12 is a top view of a third embodiment of this invention.

FIG. 13 is a side view of a means of forming the third embodiment of this invention.

FIG. 14 is a side view of the third embodiment of this invention.

FIG. 15 is a bottom view of the third embodiment of this invention.

### DETAILED DESCRIPTION OF THE INVENTION

### (1) Problems with the Prior Art

One problem that has existed in the prior art is that, when a medicinal substance is impregnated in the gauze pad, it is often wet. During storage before use, the wet medicinal substance can dry out, and be less useful than is expected. Even where a substance is impregnated in a gauze pad which will be wetted by blood or other substances exuded from the wound, this may not work well. There may not be as much moisture exuded from the wound as expected, or there may be nothing exuded.

Another problem with the prior art is that a gauze pad is not always suitable for helping a cut, wound, or a skin condition to heal quickly. For example, the common pimple on human skin is an inflamed swelling. This inflamed swelling can be relieved and minimized quickly by the application of certain substances. Some of these substances cannot be impregnated in a gauze pad and stored for a significant time.

It would be desirable if there were a way to manufacture a medicinal substance application device which can hold a dry medicinal substance for relatively long periods of time before application to a person. The medicinal substance application device would be affixed to a suitable bandage at the time of manufacture. When a person so desired, the person could apply the medicinal substance application device to a particular location on the person's body and secure it with the bandage. The person would be able to easily activate the dry medicinal substance contained within the application device, or impregnated in it, by simply wetting it with water or another liquid.

### (2) The Invention

When the particular situation calls for the application of a medicinal substance that is to be held and stored in dry or dried form, and then wetted one or more times when applied to a body, it may be desirable to hold and store the dry or dried medicinal substance in a pouch or multiple pouches. The pouch or multiple pouches can then be applied to a body, and wetted once or periodically to activate or reconstitute the medicinal substance(s) held in the pouch.

A medicament application device comprises a medical dressing means for holding a dry or dried medicinal substance, an attachment means for securely attaching the medical dressing means to a body, an aperture through the attachment means adjacent the medical dressing means, the aperture of sufficient size that a person can infuse water or another liquid through the attachment means and onto the medical dressing means. There may be a removable covering over the aperture, and the removable covering may be re-closable.

The aperture through the attachment means may be the only opening through the attachment means. Alternatively, if the attachment means has plural air holes designed to allow air to flow through the attachment means, the aperture is significantly larger than any of the air holes.

It is desirable that the medicament within the medical dressing means be distributed in a relatively uniform fashion, and that this relatively uniform distribution be maintained regardless of how the medical dressing is oriented with respect to gravity. Therefore, the medical dressing or pouch may comprise a distribution means for holding the medicament in a relatively uniformly distributed fashion within the medical dressing or pouch, and maintaining that relatively uniform distribution regardless of how the medical dressing or pouch is oriented with respect to gravity. There may be a solid, flexible baffle contained within the medical dressing such that the dry or dried medicinal substance is separated and spaced relatively uniformly throughout the medical dressing. The baffle can be a plastic matrix that, when contained within a pouch, divides the pouch into relatively small compartments. Alternatively, the pouch itself could be stamped to form plural separate compartments. When the medicament is evenly distributed among the relatively small compartments of the baffle, the medicament cannot congregate at one side or another when the pouch moves and gravity tends to pull the medicament in one direction or another. Alternatively, the medical dressing may comprise plural smaller medical dressings packed within a larger medical dressing. Utilizing plural small dressings packed within a larger dressing serves the same purpose as a baffle. Any modification to the pouch that maximizes distribution of the medicament within the pouch regardless of the pouch's orientation would come within this disclosure.

The medical dressing can be a gauze pad or a surgical dressing, or it can be a pouch. If the medical dressing is a pouch, the pouch can be completely closed or it can have a closable opening to allow the user to place a medicinal substance into the pouch and then close the opening before applying the pouch to the body.

If the medical dressing is a gauze pad, surgical dressing or the like, a medicament in a dry form can be spread or impregnated into the folds of the gauze or other material as it is being formed into a pad. The dry form of medicament could be spread throughout the pad in a relatively uniform manner, or the medicament could be concentrated in one or more particular areas in the pad, as in the center. If the medicament is in a dried form, then part or all of the pad can be wetted with a liquid or semi-liquid form of the medicament, thereby infusing the medicament into the pad, and then the pad can be allowed to dry. In both cases, subsequent wetting of the pad with water or another liquid will convert the dry or dried medicament into a liquid or semi-liquid form, or will activate or reconstitute the medicament, such that its medicinal or helpful properties are present. The wetting of the medicament in the pad should be sufficient that the medicament suffuses out of the pad and onto the part of the body which the pad is adjacent.

If the medical dressing is a pouch, the pouch can be made in any size, with any suitable material, and shaped in any suitable shape for application to a body. For example, a pouch usable as a medical dressing could be shaped like a standard tea bag and could be made of common tea bag paper, which is a combination of abaca pulp, cellulose, and sealing fibers, all of which is bleached with oxygen.

The medical dressing and the attachment means can each be formed in any suitable shape. Examples of possible shapes of the larger two dimensions (the length and width) of the medical dressing are square, rectangular, triangular, fluted, and circular. The shape could be non-symmetrical, fanciful, whimsical, or shaped particularly to fit a certain body part. It could have fingers, or it could comprise plural main parts connected by relatively thin parts. The thickness of the medical dressing could be relatively thin or relatively thick as needed. The thickness may be relatively uniform through the dressing or it could have substantially different thicknesses at different parts of the dressing. The thickness dimension may have a particular shape. The attachment means will usually be somewhat larger, and in some cases substantially larger, than the medical dressing in order to securely attach the medical dressing to its intended location. The attachment means could have plural individual medical dressings affixed to a single attachment means. The attachment means may be molded to specifically fit a certain body part. For example, an attachment means may be molded to fit a person's face or head.

The medical dressing could be made of any suitable material. Examples of possible materials with which to make the medical dressing are cotton, cloth, fabric, sponge, gel, plastic, paper, and the like. The medical dressing could be relatively stable or it could be of a substance that dissolves over time.

The medical dressing can be specifically designed to treat one or more various afflictions. Examples of common afflictions that a medical dressing could be arranged to treat are: pimples, acne, red spots, blood blisters, warts, insect stings, dry skin, sunburn, thermal burns, psoriasis, bacterial infections, and the like. The medical dressing could be designed to obtain a certain result on skin or a body part that is otherwise considered healthy. For example, a person may desire to remove dead surface skin or otherwise cause a minor skin peal to reveal younger skin. The medicinal substance could be an astringent or styptic substance. The medicinal substance could be a powder.

The medicinal substance or medicament disposed in the medical dressing can be any substance that tends to cause a remedy or a desired result. The medicinal substance or medicament can be a single substance or a combination of different substances. The medicinal substance or medicament can be solid, dry, dried, a powder, or a gel or the like. The medicinal substance or medicament can be lumpy and granular, or it could be finely grained. The medicinal substance or medicament can be placed or impregnated into the medical dressing in its natural dry state or it can be ground to a desired consistency.

The aperture through which the dry or dried medicinal substance or medicament contained in the medical dressing is wetted would need to be large enough that the entire medical dressing could be adequately wetted. The relatively small air passage holes that are commonly found in the adhesive strip of a band-aid would generally not be large enough to ensure that the entire medical dressing of the band-aid was adequately wetted. The wetting aperture would be larger. In some embodiments, the wetting aperture would be so large that a substantial portion, or even most, of the medical dressing would be visible through the aperture when any aperture covering was removed. There may be only a small portion of the medical dressing around its perimeter that actually contacts the adhesive tape in order to secure the medical dressing to the adhesive tape. The shape of the wetting aperture could be of any suitable shape to enable the entire medical dressing to be adequately wetted such that the medicinal substance or medicament within the medical dressing is also adequately wetted. The aperture's shape could be circular, square, rectangular, triangular, fluted, fanciful, whimsical, or even of a shape that provides the appearance of a symbol. The actual shape of the aperture is a design choice.

### (3) Embodiments

This invention can be embodied in multiple ways. One preferred embodiment may comprise a medicament application device that is impregnated with dried medicament. The medicament application device can be a simple gauze pad, or a surgical dressing, or the like. The medicament application device can be any useful size or thickness or shape. The medicament application device which is impregnated with the dry or dried medicament can be affixed to an adhesive strip. The adhesive strip can have an adhesive applied to one side of the strip. The adhesive strip can be any useful size, thickness, or shape, but will typically be longer and/or wider than the medicament application device in order to be able to adequately secure the medicament application device to the body. The adhesive would typically be covered by one or more non-stick strips that prevent the adhesive surface of the adhesive strip from sticking to anything until the non-stick strips are removed. The medicament application device would be affixed to the adhesive side of the adhesive strip, typically at or near the middle of the adhesive strip. The dried medicament impregnated in the medicament application device can be activated or reconstituted with water or another liquid. The water or other liquid will be applied to the medicament application device through an aperture in the adhesive strip. This aperture will typically be disposed through the adhesive strip at a place where the medicament application device is affixed to the adhesive strip. There may be a covering over the aperture such that nothing can reach the medicament application device through the aperture until the covering is removed or opened. The covering may permanently removable or it could be capable of being opened and closed multiple times. In use, the medicament application device would have a dry or dried medicament applied to or impregnated in the medicament application device. An adhesive strip will be formed in a desired shape with a suitable adhesive applied to one side of the strip. An aperture will be cut into or otherwise provided in the strip. The aperture will be disposed at the desired place on the strip and it may be covered by an openable closure. The medicament application device can be affixed to the adhesive strip at the location of the aperture, such that the medicament application device can be accessed through the aperture to apply water or another liquid to the medicament application device. The remaining adhesive area of the adhesive strip will be covered by one or more non-stick strips, and the entire assembly will be encased in an openable package. When a person desires to use the assembly, the person will open the package, and remove the assembly from the package which is then discarded. The person will then remove the non-stick strip(s) and discard them. The person will apply the medicament application device to the appropriate location on the exterior of a person's body, and affix the adhesive strip to the body in the usual manner. At this point, the medicament application device is disposed adjacent the part of the body which the person desires to medicate. The person then removes the cover covering the aperture through the adhesive strip, if there is a cover. The dry or dried medicament in the medicament application device is then wetted, typically by a person dipping a fingertip into water, or another liquid, and placing the fingertip onto the open aperture, such that the water or liquid on the fingertip travels into the medicament application device and wets the medicament therein. Sufficient water or liquid may take one or more wettings to achieve the desired amount of water or liquid in the medicament application device which will sufficiently wet the medicament therein. Of course, water or another liquid can be applied to the medicament application device through the opened aperture in any suitable way, and not just with a fingertip. Any means of introducing water or another liquid through the aperture and into the medicament application device will fall within this disclosure. After introduction of a sufficient amount of water or another liquid through the aperture, the aperture's cover can be replaced or the aperture can be left open as is desired. It may be necessary to re-wet the medicament application device at one or more later times, to keep the medicament from becoming too dry. This embodiment can be made in any shape and size. The medicament can be any medicinal substance that is dry or capable of being dried, and which can then be later wetted to provide a medicinal property. Medicinal substances can comprise relatively common substances such as sea salt, tea, aloe, or herbs or combinations thereof. One medicament application device may contain just one medicament, or it could contain plural different medicaments. The medicament(s) may be contained within the medicament application device and/or suffused and/or impregnated with the medicament.

### (4) Description of the Drawings

FIG. 1 shows a first preferred embodiment of this invention. In FIG. 1, shows a band-aid 10 comprising an adhesive strip 15 which has a gauze pad 20 affixed to a first side 25 of the adhesive strip 15. The gauze pad 20 can hold a dry or dried medicament or medicinal substance. The gauze pad 20 affixed to a first side 25 of the adhesive strip 15 covers a portion of the first side 25 of the adhesive strip 15. The remainder of the first side 25 has an adhesive 35 disposed on that side. The opposite second side 30 of the adhesive strip 15 has no adhesive. There is an aperture 40 completely through the adhesive strip 15, thereby exposing the gauze pad 20 from the second side 30. The aperture 40 through the adhesive strip 15 is disposed generally in a location centered over the gauze pad 20. FIG. 2 is a top view of the first preferred embodiment. The second side 30 of the adhesive strip has the aperture 40 through which one can see the gauze pad 20. FIG. 3 is a bottom view of the first preferred embodiment. The first side 25 of the adhesive strip has affixed on it the gauze pad 20 in a central location. The remaining portions of the first side 25 of the gauze pad have disposed on them an adhesive 35. In use, a person would apply the band-aid 10 to an appropriate site on a person's body, and then cause water or another liquid to be passed through the aperture 40 and into the gauze pad 20 in sufficient quantity to wet substantially all the gauze pad. This could be done by wetting a fingertip and putting the fingertip to the aperture 40, such that the liquid that is on the fingertip drips or passes onto the gauze pad. Alternatively, a cotton swab, an eyedropper, or a similar instrument could be used instead of a fingertip to pass a liquid through the aperture 40 and into the gauze pad 20.

FIG. 4 shows an alternative embodiment of this invention. FIG. 4 shows the same band-aid 10 as shown in FIGS. 1 to 3. However, in FIG. 4, there is shown a pull-tab cover adhesively attached to the second side 30 of the adhesive strip 15, and completely covering the aperture 40 shown in FIG. 2. When a person desires to use the band-aid 10 shown in FIG. 4, the person will first apply the band-aid 10 to an appropriate site on a body, and then remove the pull-tab 45 cover, thereby exposing the gauze pad 20 through the aperture 40. The person can then wet the gauze pad 20 through the aperture 40 as previously described.

FIG. 5 shows another alternative embodiment of this invention. FIG. 5 shows the same band-aid 10 as shown in FIGS. 1 to 3. However, in FIG. 5, there is a portion 50 of the adhesive strip 15 that has been perforated 55 in a circular pattern such that a user can grasp the perforated portion 50 with the fingers and easily pull that portion 50 away from the adhesive strip 15, thereby creating an aperture through the adhesive strip 15 similar to the aperture 40 shown in FIG. 2. The round perforated portion 50 would normally be disposed of after being pulled away.

The first preferred embodiment is shown and described as a band-aid with a rectangular gauze pad. But this embodiment is not limited to a band-aid and a gauze pad. The drawings showing the first embodiment can also be described as showing a bandage with a surgical dressing or any medical dressing. The size and shape of the devices shown in the figures is not limited and the devices can be applied to accomplish any purpose a bandage can accomplish if suitably sized or shaped. The medical dressing could be any size or shape that accomplishes the purpose of applying a medicament to a body, and the attachment means could be any size or shape that accomplishes the purpose of attaching the medical dressing to the body and allowing a liquid to be passed through the attachment means to wet the medical dressing and its medicament.

FIG. 6 shows a second preferred embodiment of this invention. In FIG. 6, a band-aid 60 comprises an adhesive strip 65 and a pouch 70 affixed to a first side 75 of the adhesive strip 65. The pouch 70 affixed to a first side 75 of the adhesive strip 65 covers a portion of the first side 75 of the adhesive strip 65. The remainder of the first side 75 of the adhesive strip 65 has an adhesive 85 disposed on that side. The opposite second side 80 of the adhesive strip 65 has no adhesive. FIG. 7 shows a top view of the band-aid 60 of FIG. 6. There is an aperture 90 completely through the adhesive strip 65. The aperture is disposed generally in a location centered over the pouch 70. FIG. 8 shows a bottom view of the band-aid 60, and shows the pouch 70 disposed in a generally central location with adhesive disposed around the remainder of the first side 75 of the adhesive strip. A dry or dried medicinal substance is contained within the pouch 70. The pouch 70 is made of a material that is permeable to liquid and is sufficiently strong to contain the enclosed medicament even when the pouch is wet. The pouch may be sealed closed, or it may have a closable opening so that medicament can be added.

To make the pouch 70, one can cut two sheets of pouch material about the same size and shape. One would then place the first sheet over a forming device that had a depressed area of the proper size and shape. The flexible first sheet will naturally form a depressed area in the depressed area of the forming device. A dry or dried medicament would be placed onto the first sheet in the depressed place. The second sheet would then be placed over the first sheet, and the perimeter of the two sheets sealed together to completely contain the medicinal substance between them. Alternatively, one sheet could be used and folded over itself, as is well-known in this art. Tea and salt are commonly packaged in this manner.

The aperture 90 through the adhesive strip 65 in this second embodiment will be sized such that most of the pouch 70 is exposed through the aperture 90. Enough of the pouch 70 will be affixed all around the perimeter such that the pouch 70 is securely affixed to the adhesive strip 65. An aperture 90 of this size will help ensure that all of the pouch 70 can be adequately wetted through the aperture 90. Wetting can be accomplished by transferring a liquid with a finger, swab, eyedropper, or other suitable device. Liquid can also be poured directly onto the pouch 70 through the aperture 90 from a bottle or other container.

Sufficient dry or dried medicament may be packed into the pouch 70 to completely fill it. However, if the dry or dried medicament loses volume when wetter, or if insufficient medicament is packed into the pouch to completely fill it, then the medicament may shift inside the pouch due to gravity when placed on the body in use. To minimize shifting of the medicament within the pouch in use, a flexible baffle defining plural compartments can be placed into the pouch during formation and the medicament distributed among all compartments. This is shown in FIG. 10. Pouch 70 is shown containing a baffle 100. The medicament 105 is distributed in a relatively uniform way among the compartments in the baffle such that if the pouch 70 changes direction, the medicament 105 remains relatively uniformly distributed through the pouch 70. Once the pouch is closed and sealed, the medicament will tend not to shift too much due to the baffle. Pouch 70 in FIG. 10 is shown with a large opening 110 in the pouch to illustrate the baffle 100 and medicament 105, but the pouch would normally be completely closed in use.

Alternatively, as is shown in FIG. 11, the dry or dried medicament can be packed into plural small sealed pouches 115, and the plural small pouches 115 packed into a larger pouch 70. The larger pouch 70 is then affixed to the adhesive strip 65 as described above. The plural small pouches 115 tend to minimize shifting of the medicament due to gravity during use. The pouch 70 in FIG. 11 is shown with a large opening 110 which would normally not be present in use, but is presented here to illustrate the plural pouches 115 within the pouch 70.

FIGS. 12 to 15 show a third preferred embodiment of this invention, and one way it can be made. FIG. 12 shows two separate pieces of bandage material from which this band-aid can be made. Both pieces of bandage material can be made from any known bandage material, but are preferably cotton fabric. The first piece 120 is somewhat larger than the second piece 125, but the exact size and shapes of each piece are a matter of design choice.

To make the band-aid, the first piece 120 is laid down onto a jig 130 that is generally flat except for a sunken concave area 135 that is sized to be somewhat smaller both in length and width than the second piece 125 of bandage material. As the first piece 120 is laid upon the jig 130, it may naturally sink down into the sunken area 135 of the jig 130 due to the flexibility of the first piece 120, or it may be necessary to apply heat and/or moisture to relax the first piece 120 sufficiently for it to generally sink into the concavity of the sunken area 135. Alternatively, the first piece 120 could be pushed into the sunken area 135 by a finger or a convex jig piece (not shown).

When the first piece 120 of bandage material has been laid into the jig 130, a medicament 140 can then be placed onto the first piece 120 in the sunken area of the first piece 120 as is shown in FIG. 13. The sunken area can be filled with medicament 140 to the level of the jig's flat surface, or overfilled somewhat. However, overfilling such that medicament 140 spills to the sides of the sunken area should be avoided to prevent interference with seals. Once, the medicament 140 has been placed onto the first piece 120, the second piece 125 is aligned above the medicament 140 as shown in FIG. 13 and brought down on top of the first piece 120 and the medicament 140. The edges 145 around the periphery of the medicament 140 are then sealed, either with heat or adhesive or another suitable sealing means.

After sealing the second piece 125 of bandage material to the first piece 120, the medicament 140 is securely contained between the two pieces of bandage material, as is shown in FIG. 14. There remain areas 150 on each side of the second piece 125 of bandage material where the first piece 120 of bandage material remains exposed. An adhesive can be applied to these remaining areas. If desired, a non-stick tab (not shown) can be applied over the two adhesive areas to prevent them from sticking to anything until the bandage is applied by the intended user. The completed bandage can then be removed from the jig 130 and packaged as desired.

FIG. 15 shows one side of the finished bandage, that side being the one on which the second piece 125 lies on the first piece 120. In FIG. 15, one sees the area 145 around the medicament where the second piece is sealed to the first piece. One also sees the two areas 150, one on each side of the medicament, where adhesive has been applied to one side of the first piece of bandage material.

The first piece 120 of bandage material and the second piece 125 of bandage material can be made of the same material or different materials. The adhesive can alternatively be applied to the opposite side of the first piece than that described above.

An alternative to this embodiment would be to place the smaller piece on the jig, place the medicament on it, then place the larger piece over the smaller piece and seal the two pieces around the perimeter of the medicament. The adhesive can then be applied as described above. Another alternative would be to utilize two pieces of approximately the same size. The perimeter where the adhesive is applied would be two membranes and the perimeter would be somewhat stiffer. Yet another alternative embodiment would be to utilize only one piece of material that is folded over to make two sides.

In all embodiments and in all alternatives, the location of the adhesive used to attach the bandage to the body is a matter of design choice. The location(s) chosen only need to adequately secure the bandage to the body. However, it may be desirable to leave an opening between the means for attaching the medical dressing or pouch and the body at some location so that liquid can escape from the means for attaching. When the user puts water through the aperture of the attachment means and into the medical dressing or pouch, the escape of liquid from the attachment means will signal to the user that enough liquid has been infused into the medical dressing or pouch to activate or reconstitute the medicament. The amount and location of any opening between the attachment means and the body is a matter of design choice. The opening can be simply an area left unattached or there could be an opening incorporated into the attachment means which is specifically designed to allow excess liquid to escape.

A person of ordinary skill who reads the disclosure of this embodiment and these two alternatives will be able to design different alternative to the ones enumerated here, all of which will come within the scope of this invention.

This bandage embodiment is simple, quick, and inexpensive to manufacture, and it uses the minimum amount of bandage materials to accomplish its intended purpose.

The description of the invention and of the preferred embodiments will enable persons of ordinary skill in this art to devise other embodiments that come within the scope of this invention. This patent application is meant to be limited only by the following claims.

I claim:

1. A medicament application device comprises:
1) a medical dressing means for holding a dry or dried medicinal substance;
2) the medical dressing affixed to an attachment means for securely attaching the medical dressing means to a body;
3) an aperture through the attachment means adjacent the medical dressing means, the aperture of sufficient size that a person can infuse a liquid through the attachment means and onto the medical dressing means; and
4) the medical dressing containing a dry or dried medicament.

2. The device of claim 1 further comprising a removable covering disposed over the aperture.

3. The device of claim 2 wherein the removable covering is a pull-tab.

4. The device of claim 2 wherein the removable covering is a perforated section of the attachment means.

5. The device of claim 1 further comprising a means associated with the medical dressing for causing the medicament to remain relatively uniformly distributed throughout the medical dressing regardless of how the medical dressing is oriented with respect to gravity.

6. The device of claim 1 wherein the medical dressing is a liquid-permeable pouch with a baffle enclosed within the pouch, and wherein the medicament is contained within the pouch.

7. The device of claim 1 wherein the medical dressing is a first relatively large liquid-permeable pouch with plural relatively small pouches contained within the first pouch and wherein each of the plural relatively small pouches contains part of the medicament.

8. A bandage comprising:
1) a pouch containing a dry or dried medicament, the pouch made of a material that is liquid-permeable; and
2) an attachment means for removably attaching the pouch to a location on a body to which the medicament is to be delivered;
3) the attachment means having an aperture through the attachment means at a location adjacent the pouch.

9. The bandage of claim 8 wherein the attachment means is a strip of adhesive tape.

10. The bandage of claim 8 further comprising a removable covering disposed over the aperture through the attachment means on the side of the attachment means opposite the pouch.

11. The device of claim 10 wherein the removable covering is a pull-tab.

12. The device of claim 10 wherein the removable covering is a perforated section of the attachment means.

13. The device of claim 8 further comprising a means associated with the pouch for causing the medicament in the pouch to remain relatively uniformly distributed throughout the pouch regardless of how the pouch is oriented with respect to gravity.

14. The device of claim 8 wherein the liquid-permeable pouch comprises a baffle enclosed within the pouch, and wherein the medicament is contained within the pouch and spread relatively uniformly throughout the baffle.

15. The device of claim 8 wherein the liquid-permeable pouch is a first relatively large pouch with plural relatively small pouches contained within the first pouch, and wherein each of the plural relatively small pouches contains part of the medicament.

16. A bandage comprising:
1) a pouch containing a dry or dried medicament, the pouch made of a material that is liquid-permeable; and
2) an attachment means for removably attaching the pouch to a location on a body to which the medicament will be delivered;
3) the attachment means having an aperture through which the pouch can be wetted by a liquid.

17. The bandage of claim 16 wherein the attachment means is a strip of tape having adhesive disposed over at least part of one side of the tape.

18. A method of delivering a medicament to a desired location on a body, comprising the steps of:
1) disposing a dry or dried medicament in a liquid-permeable pouch;
2) affixing the pouch to an attachment means for attaching the pouch to the desired location on the body;
3) attaching the pouch to the location on the desired location on the body with the attachment means; and
4) wetting the pouch through an aperture in the attachment means with an amount of liquid sufficient to wet the medicament in the pouch.

19. The method of claim 18 further comprising the step of:
5) continuing to wet the pouch through the aperture until some liquid is observed escaping from between the attachment means and the body.

20. The method of claim 18 further comprising the step of:
5) waiting a period of time after wetting the pouch, and then re-wetting the pouch with an amount of liquid sufficient to re-wet the medicament in the pouch through the aperture in the attachment means.
